(19) United States
(12) Patent Application Publication
(10) Pub. No.: US 2020/0201077 A1
(43) Pub. Date: Jun. 25, 2020

(54) ULTRASOUND COMMUNICATION BETWEEN OPHTHALMIC LENSES THROUGH THE EYE

(71) Applicant: Johnson & Johnson Vision Care, Inc., Jacksonville, FL (US)

(72) Inventors: Donald Scott Langford, Melbourne, FL (US); Adam Toner, Jacksonville, FL (US)

(21) Appl. No.: 16/227,947

(22) Filed: Dec. 20, 2018

(51) Int. Cl. G02C 7/08; G08C 23/02; A61F 2/16

(52) U.S. Cl. CPC G02C 7/083; A61F 2250/0002; A61F 2/16; G08C 23/02

(57) ABSTRACT

A pair of ophthalmic lens having an electronic system is described herein for communicating between them using ultrasound transducers for creating a sound pressure wave(s) propagated through the eyes and the optic nerve of the wearer of the contact lenses. The ophthalmic lenses include at least one ultrasound module having at least one transducer such as a pair of transmit and receive transducers and a transceiver transducer that are optic nerve facing. The ultrasound module includes additional components for the transmission and receipt of the sound pressure wave(s). In an alternative embodiment, there is one ultrasound module with a multiplexer connected to a plurality of transducers. In at least one embodiment, the sound pressure wave(s) encodes a message between the ophthalmic lenses.

### BACKGROUND OF THE INVENTION

### 1. Field of the Invention

The present invention relates to a powered or electronic ophthalmic lens, and more particularly, to a powered or electronic ophthalmic lens having an ultrasound module to provide a communication link through the eyes and the optical nerve of the wearer.

### 2. Discussion of the Related Art

As electronic devices continue to be miniaturized, it is becoming increasingly more likely to create wearable or embeddable microelectronic devices for a variety of uses. Such uses may include monitoring aspects of body chemistry, administering controlled dosages of medications or therapeutic agents via various mechanisms, including automatically, in response to measurements, or in response to external control signals, and augmenting the performance of organs or tissues. Examples of such devices include glucose infusion pumps, pacemakers, defibrillators, ventricular assist devices and neurostimulators. A new, particularly useful field of application is in ophthalmic wearable lenses and contact lenses. For example, a wearable lens may incorporate a lens assembly having an electronically adjustable focus to augment or enhance performance of the eye. In another example, either with or without adjustable focus, a wearable contact lens may incorporate electronic sensors to detect concentrations of particular chemicals in the precorneal (tear) film. The use of embedded electronics in a lens assembly introduces a potential requirement for communication with the electronics, for a method of powering and/or re-energizing the electronics, for interconnecting the electronics, for internal and external sensing and/or monitoring, and for control of the electronics and the overall function of the lens.

The human eye has the ability to discern millions of colors, adjust easily to shifting light conditions, and transmit signals or information to the brain at a rate exceeding that of a high-speed internet connection. Lenses, such as contact lenses and intraocular lenses, currently are utilized to correct vision defects such as myopia (nearsightedness), hyperopia (farsightedness), presbyopia and astigmatism. However, properly designed lenses incorporating additional components may be utilized to enhance vision as well as to correct vision defects.

Conventional contact lenses are polymeric structures with specific shapes to correct various vision problems as briefly set forth above. To achieve enhanced functionality, various circuits and components have to be integrated into these polymeric structures. For example, control circuits, microprocessors, communication devices, power supplies, sensors, actuators, light-emitting diodes, and miniature antennas may be integrated into contact lenses via custom-built optoelectronic components to not only correct vision, but to enhance vision as well as provide additional functionality as is explained herein. Electronic and/or powered ophthalmic lenses may be designed to provide enhanced vision via zoom-in and zoom-out capabilities, or just simply modifying the refractive capabilities of the lenses. Electronic and/or powered contact lenses may be designed to enhance color and resolution.

The proper combination of devices could yield potentially unlimited functionality; however, there are a number of difficulties associated with the incorporation of extra components on a piece of optical-grade polymer. In general, it is difficult to manufacture such components directly on the lens for a number of reasons, as well as mounting and interconnecting planar devices on a non-planar surface. It is also difficult to manufacture to scale. The components to be placed on or in the lens need to be miniaturized and integrated onto just 1.5 square centimeters of a transparent polymer while protecting the components from the liquid environment on the eye. It is also difficult to make a contact lens comfortable and safe for the wearer with the added thickness of additional components.

In addition, because of the complexity of the functionality associated with a powered lens and the high level of interaction between all of the components comprising a powered lens, there is a need to coordinate and control the overall operation of the electronics and optics comprising a powered ophthalmic lens. Accordingly, there is a need for a system to control the operation of all of the other components and provide communication between the contact lenses that is safe, low-cost, and reliable, has a low rate of power consumption and is scalable for incorporation into an ophthalmic lens.

There are several scenarios where there is a need for powered contact lenses to communicate during normal operation. Methods of detecting and changing lens state for presbyopia, commonly referred to as accommodation, may require the state of the left and right eye to be shared to determine if the lens focus should be changed. In each case, the independent state of each eye must be communicated so that the system controller can determine the required state of the variable lens actuator. There are other cases where it may enhance the user experience if the lens state (e.g., focus state) is changed in a coordinated fashion.

### SUMMARY OF THE INVENTION

Lens-to-lens communication may take place wirelessly. There at least two known approaches to communicate wirelessly at a scale appropriate for an ophthalmic lens application. These approaches are photonic (light) and radio frequency (RF) communication. Photonic communication, or communication using light, is difficult because the power consumption associated with generating photonic signals sufficiently powerful to overcome ambient interference may be not be possible to achieve using a power source appropriate for use in an ophthalmic lens device. RF communication may be possible, however it presents in own challenges. Among other issues, antennas dimensioned to fit within a typical ophthalmic lens device require quite higher than typical RF frequencies in order to operate. Generating these higher frequency signals requires more power. Coupled with the fact that RF energy is absorbed by human tissue, thus reducing power at the receiver, the practical application of RF communication in an ophthalmic lens device may not be feasible.

Accordingly, there remains a need for an improved wireless communication method and device that is capable of safely and reliably transmitting signals from and to ophthalmic lens devices, such as between contact lenses disposes on the eyes of a wearer. The devices and methods disclosed and claimed here utilize ultrasound communication. Ultrasound communication is desirable for many reasons, including that the sound spectrum is unregulated and there are few interfering background signals. Furthermore, ultrasound frequencies are orders of magnitude lower than required RF frequency for a similar application. Accordingly, the power levels required to generate ultrasound signals are therefore lower than alternative signals, such as RF, for a similar application. Further still, ultrasound energy has significantly less absorption in the human body, which necessitates much less power and allows the user of lower and safer levels of energy in the eye of the wearer. Embodiments of the invention disclosed herein illustrate novel technical implementations of an ophthalmic device configured to enable ultrasound communication in an effective, safe, and power-effective manner.

In at least one embodiment, an ophthalmic lens system includes: a first ophthalmic lens; a second ophthalmic lens; and where each ophthalmic lens having an ultrasound module in the ophthalmic lens, the ultrasound module includes at least one ultrasound transducer optic nerve facing and orientated such that when a sound pressure wave is produced by the at least one transducer, the sound pressure wave travels through an eye on which the ophthalmic lens is located to an optical nerve connected to the eye, and a system controller in electrical communication with the ultrasound module, the system controller configured to provide a control signal to the ultrasound module where the control signal includes a message to be transmitted by the ultrasound module, the system controller configured to receive an output from the ultrasound module and to perform a function in response to a receive message embodied in the output. . . . In a further embodiment to the previous embodiment, the ultrasound module on the first ophthalmic lens configured to produce the sound pressure wave at a first frequency, the ultrasound module on the second ophthalmic lens configured to produce the sound pressure wave at a second frequency, the ultrasound module on the second ophthalmic lens has a receive transducer tuned to sense the sound pressure wave at the first frequency, the receive transducer is optic nerve facing, and the ultrasound module on the first ophthalmic lens has a receive transducer tuned to sense the sound pressure wave at the second frequency, the receive transducer is optic nerve facing. In a further embodiment to the previous embodiment, the ultrasound module on the first ophthalmic lens has a second receive transducer tuned to sense the sound pressure wave at the first frequency, and the ultrasound module on the second ophthalmic lens has a second receive transducer tuned to sense the sound pressure wave at the second frequency, and the second receive transducers are optic nerve facing. In a further embodiment to any of the above embodiment, the ultrasound modules are near a perimeter of the respective ophthalmic lens. For the purpose of this disclosure, near the perimeter may be considered as being within the non-optic zone (i.e., peripheral zone) and within approximately the outer twenty percent of the radial width of the lens in which the ultrasound module is disposed.

In a further embodiment to any of the above embodiments, the at least one transducer includes a transmit transducer and a receive transducer, and each ultrasound module includes a processor in electrical communication with the system controller; a transmit path having an oscillator in electrical communication with the processor, a burst generator in electrical communication with the oscillator and the processor, a transmit driver in electrical communication with the burst generator configured to receive a burst signal from the burst generator, the transmit transducer in electrical communication with the transmit driver; and at least one receive path having the receive transducer, a receive amplifier in electrical communication with the receive transducer and configured to amplify an output of the receive transducer, and an analog signal processor in communication with the receive amplifier and the processor, and where the processor configured to control whether the transmit path and the at least one receive path are activated. In a further embodiment to the previous embodiment, each ultrasound module includes two receive paths, the two receive paths having the receive transducer tuned to different frequencies. In a further embodiment to any of the above embodiments of the previous paragraph, the at least one transducer includes a plurality of transducers, and the ultrasound module includes a processor in electrical communication with the system controller; a multiplexer in electrical communication with the plurality of transducers; a transmit path having an oscillator in electrical communication with the processor, a burst generator in electrical communication with the oscillator and the processor, a transmit driver in electrical communication with the burst generator configured to receive a burst signal from the burst generator and the multiplexer; and at least one receive path having a receive amplifier in electrical communication with the multiplexer and configured to amplify an output of the receive transducer, and an analog signal processor in communication with the receive amplifier and the processor, and wherein the processor configured to control whether the transmit path and the at least one receive path are activated, and the multiplexer provides selective communication between at least one transducer with the transmit path or the at least one receive path. In a further embodiment to any of the above embodiments of the previous paragraph, the at least one transducer includes one transducer, and each ultrasound module includes a processor in electrical communication with the system controller; the transducer; a switch in electrical communication with the processor; a transmit path having an oscillator in electrical communication with the processor, a burst generator in electrical communication with the oscillator and the processor, a transmit driver in electrical communication with the burst generator configured to receive a burst signal from the burst generator, the transmit driver drives the transducer when connected through the switch; and at least one receive path having a receive amplifier in electrical communication with the transducer through the switch and configured to amplify an output of the transducer, and an analog signal processor in communication with the receive amplifier and the processor, and where the processor configured to control whether the transmit path and the at least one receive path are activated based on an operation mode of the ultrasound module between transmit and receive, and the processor configured to control the switch and the operation mode. In a further embodiment to any of the above embodiments of the previous paragraph, each ophthalmic lens further includes a power source in electrical communication with the system controller and the ultrasound module; the at least one transmitter includes a transmit transducer and a receive transducer; and each ultrasound module includes a processor in electrical communication with the system controller; a transmit path having an oscillator in electrical communication with the processor, a pulse generator in electrical communication with the oscillator and the processor, a charge pump in electrical communication with the power source, a transmit driver in electrical communication with the pulse generator and the charge pump, the transmit driver configured to receive a signal from the pulse generator, the transmit transducer in electrical communication with the transmit driver; and at least one receive path having the receive transducer, a receive amplifier in electrical communication with the receive transducer and configured to amplify an output of the receive transducer, and an envelope detector in electrical communication with the receive amplifier, an analog signal processor in communication with the envelope detector and the processor, and where the processor configured to control whether the transmit path and the at least one receive path are activated. In a further embodiment to any of the above embodiments of the previous paragraph, each ophthalmic lens further includes a power source in electrical communication with the system controller and the at least one ultrasound module; the at least one transmitter includes a transmit transducer and a receive transducer; and each ultrasound module includes a processor in electrical communication with the system controller; a transmit path having an oscillator in electrical communication with the processor, an amplitude modulation modulator in electrical communication with the oscillator and the processor, a charge pump in electrical communication with the power source, a transmit driver in electrical communication with the amplitude modulation modulator and the charge pump, the transmit driver configured to receive a signal from the amplitude modulation modulator, the transmit transducer in electrical communication with the transmit driver; and at least one receive path having the receive transducer, a receive amplifier in electrical communication with the receive transducer and configured to amplify an output of the receive transducer, and an envelope detector in electrical communication with the receive amplifier, an analog signal processor in communication with the envelope detector and the processor, and wherein the processor configured to control whether the transmit path and the at least one receive path are activated.

In at least one embodiment, a method for facilitating communication between a first ophthalmic lens and a second ophthalmic lens when being used by a person where each ophthalmic lens includes at least one ultrasound module in electrical communication with a system controller, the ultrasound modules having an iris-facing transmit transducer, the method including: sending a control signal from the system controller on the first ophthalmic lens to the ultrasound module on the first ophthalmic lens where the control signal embodies a message intended for the second ophthalmic lens; preparing an output signal by the ultrasound module on the first ophthalmic lens based on the message; driving the transmit transducer on the first ophthalmic lens based on the output signal to produce at least one sound pressure wave directed at the eye to travel through an optical nerve to the other eye; receiving with a transducer on the second ophthalmic lens the sound pressure wave from the transducer on the first ophthalmic lens transmitted through the optical nerve; converting with the ultrasound module on the second ophthalmic lens an analog signal produced by the transducer on the second ophthalmic lens in response to the received sound pressure wave; providing an output to the system controller on the second ophthalmic lens from the ultrasound module on the second ophthalmic lens; and converting with the system controller on the second ophthalmic lens the output into the message from the system controller on the first ophthalmic lens, and where the optical nerve and the eyes of the person wearing the ophthalmic lenses provides a medium through which the sound pressure wave travels between the first ophthalmic lens and the second ophthalmic lens. In a further embodiment to the previous embodiment, the method further including: sending a control signal from the system controller on the second ophthalmic lens to the ultrasound module on the second ophthalmic lens where the control signal embodies a message intended for the first ophthalmic lens; preparing an output signal by the ultrasound module on the second ophthalmic lens, where the output signal embodies the message for the first ophthalmic lens based on the message intended for the first ophthalmic lens; driving the transmit transducer on the second ophthalmic lens based on the output signal to produce at least one sound pressure wave directed at the eye to travel through an optical nerve to the other eye; receiving with a receive transducer on the first ophthalmic lens sound pressure wave from the transducer on the second ophthalmic lens transmitted through the optical nerve; converting with the ultrasound module on the first ophthalmic lens an analog signal produced by the receive transducer on the first ophthalmic lens; providing an output to the system controller on the first ophthalmic lens from the ultrasound module on the first ophthalmic lens; and converting with the system controller on the first ophthalmic lens the output into the message from the system controller on the first ophthalmic lens, and where the optical nerve and the eyes of the person wearing the ophthalmic lenses provides the medium through which the sound pressure wave travels between the second ophthalmic lens and the first ophthalmic lens.

In a further embodiment to the previous embodiments, the sound pressure waves produced by the first and second ophthalmic lens are at different frequencies. In a further embodiment to the above method embodiments, each ultrasound module includes the receive transducer tuned to the frequency of the transmit transducer of the other ophthalmic lens and a second receive transducer tuned to the frequency of the transmit transducer of its ophthalmic lens.

In a further embodiment to the previous method embodiments, the method of operation of one ophthalmic lens further including: sampling the receive transducer at a first sampling rate; sampling the receive transducer at a second sampling rate when the sound pressure wave is detected by the system controller based on the output from the receive transducer; determining with the system controller when the message being received is complete; and sampling the receive transducer at the first sampling rate when the system controller determines completion of the message being sent.

In a further embodiment to the previous method embodiments, the method further including deactivating the transmission components of the ultrasound module when not transmitting. In a further embodiment to the previous method embodiments, the message sent by the system controller of the first ophthalmic lens uses a predefined protocol.

In a further embodiment to the previous method embodiments, the message sent by the system controller of the first ophthalmic lens includes instructions for the second ophthalmic lens to perform a predefined function. In a further embodiment to the previous embodiment, the predefined function includes providing a sensor reading where the sensor reading will be used by the system controller on the first ophthalmic lens to confirm interpretation of at least one sensor reading on the first ophthalmic lens; and the method further including the system controller on the second ophthalmic lens sending the sensor reading in a message provided to the respective ultrasound module, and producing a sound pressure wave with the transmit transducer of the second ophthalmic lens applied to the eye on which the second ophthalmic lens is located where the sound pressure wave travels through the optical nerve to the first ophthalmic lens.

In a further embodiment to the previous method embodiments, the message sent by the system controller of the first ophthalmic lens includes sensor readings from at least one sensor on the first ophthalmic lens. In a further embodiment to the previous method embodiments, the message is a wake-up message to activate the ultrasound module on the second ophthalmic lens.

In a further embodiment to any of the above embodiments, the first ophthalmic lens is a first contact lens and the second ophthalmic lens is a second contact lens; or the first ophthalmic lens is a first intraocular lens and the second ophthalmic lens is a second intraocular lens.

Further to the previous embodiments, the ophthalmic lens includes an intraocular lens and/or a contact lens.

Further to any of the embodiments above, a message sent by the system controller of the first ophthalmic lens uses a predefined protocol. Further to any of the embodiments above, the message sent by the system controller of the first ophthalmic lens includes instructions for the second ophthalmic lens to perform a predefined function. Further to any of the embodiments above, the message sent by the system controller of the first ophthalmic lens includes sensor readings from at least one sensor on the first ophthalmic lens.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 illustrates a contact lens having at least one ultrasound module in accordance with at least one embodiment of the present invention.

FIG. 2 illustrates a contact lens having at least one ultrasound module and a system controller having a register in accordance with at least one embodiment of the present invention.

FIG. 3 illustrates a contact lens having at least one ultrasound module and a timing circuit in accordance with at least one embodiment of the present invention.

FIG. 4 illustrates an ultrasound module in accordance with at least one embodiment of the present invention.

FIG. 5 illustrates an ultrasound module with one transducer and a multiplexer in accordance with at least one embodiment of the present invention.

FIG. 6 illustrates an ultrasound module with two receive transducers in accordance with at least one embodiment of the present invention.

FIG. 7 illustrates an ultrasound module with a charge pump, a pulse generator, and an envelope detector in accordance with at least one embodiment of the present invention.

FIG. 8 illustrates an ultrasound module with a charge pump, an amplitude modulator, and an envelope detector in accordance with at least one embodiment of the present invention.

FIG. 9 illustrates a partial cross-section of a human head wearing a pair of contact lenses in accordance with at least one embodiment of the present invention.

FIG. 10. illustrates a diagrammatic representation of an electronic insert, including a pair of transducers, for a powered contact lens in accordance with at least one embodiment of the present invention.

FIG. 11 illustrates a diagrammatic representation of an electronic insert, including a transducer, for a powered contact lens in accordance with at least one embodiment of the present invention.

FIG. 12 illustrates a diagrammatic representation of evenly spaced ultrasound modules/transducers in accordance with at least one embodiment of the present invention.

FIG. 13 illustrates an ultrasound module with a plurality of transmit/receive transducer pairs or transceiver transducers in accordance with at least one embodiment of the present invention.

FIG. 14 illustrates a communication method for two contact lenses in accordance with at least one embodiment of the present invention.

FIG. 15 illustrates a communication method for two contact lenses in accordance with at least one embodiment of the present invention.

### DETAILED DESCRIPTION OF THE EMBODIMENTS

Conventional contact lenses contain polymeric structures with specific shapes to correct various vision problems as briefly set forth above. To achieve enhanced functionality, various circuits and components may be integrated into these polymeric structures. For example, control circuits, microprocessors, communication devices, power supplies, sensors, ultrasound modules, and miniature antennas may be integrated into contact lenses via custom-built optoelectronic components to not only correct vision, but to enhance vision as well as provide additional functionality as is explained herein. Electronic and/or powered contact lenses may be designed to provide enhanced vision via zoom-in and zoom-out capabilities, or just simply modifying the refractive capabilities of the lenses. Electronic and/or powered contact lenses may be designed to enhance color and resolution.

The powered or electronic contact lens in at least one embodiment may include a variable-focus optic component and may further contain embedded electronics that enable any suitable functionality. The electronic devices consistent with the present invention may be incorporated into any number of contact lenses as described above. In addition, intraocular lenses may also incorporate various components and functionality described herein. For purposes of this disclosure, an ophthalmic lens is defined as including contact lenses, intraocular lenses, corneal onlay, corneal inlays, and the like. However, for ease of explanation, the disclosure will focus on an electronic contact lens appreciating that one of skill in the art, upon reading this disclosure, would understand how to apply its teachings to other ophthalmic device applications.

The present invention may be employed in a powered ophthalmic device having an electronic system, which actuates a variable-focus optic or any other device or devices configured to implement any number of numerous functions that may be performed. The electronic system may include one or more batteries or other power sources, power management circuitry, one or more sensors, clock generation circuitry, control algorithms and circuitry, and lens driver circuitry. The complexity of these components may vary depending on the required or desired functionality of the lens.

Control of an electronic or a powered ophthalmic device may be accomplished through a manually operated external device that communicates with the lens through ultrasonic communication, such as a hand-held remote unit, a phone, a storage container, spectacles, glasses, or a cleaning box. For example, an external device may communicate wirelessly using ultrasound with the powered ophthalmic lens based upon manual input from the wearer. Alternatively, control of the powered ophthalmic lens may be accomplished via feedback or control signals directly from the wearer. For example, the ultrasound modules in at least one embodiment may include a transmit ultrasound transducer and at least one receive ultrasound transducer, a combination transmit/receive ultrasound transducer, or a combination passive transmit/receive ultrasound transducer.

Because of the complexity of the functionality associated with a powered ophthalmic lens and the high level of interaction between components comprising a powered lens, it may be beneficial to coordinate and control the overall operation of the electronics and optics comprising a powered ophthalmic lens. Accordingly, there is a need for a system to control the operation of components and provide communication between power ophthalmic lenses that is low-cost and reliable, has a low rate of power consumption, and is scalable for incorporation into an ophthalmic lens.

In at least one embodiment, a sound pressure wave that is produced at the transmit ultrasound transducer propagates from the contact lens into the eye to provide communication between the contact lenses. In at least one embodiment, the sound pressure wave includes a burst or multiple sound pressure waves. In at least one embodiment, the sound pressure wave is used for communication.

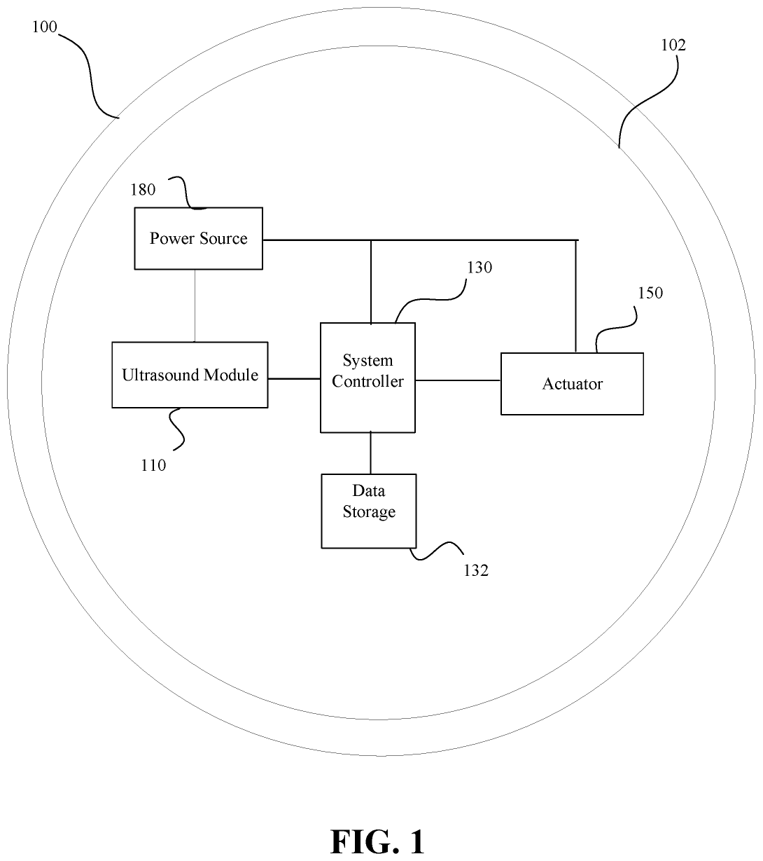

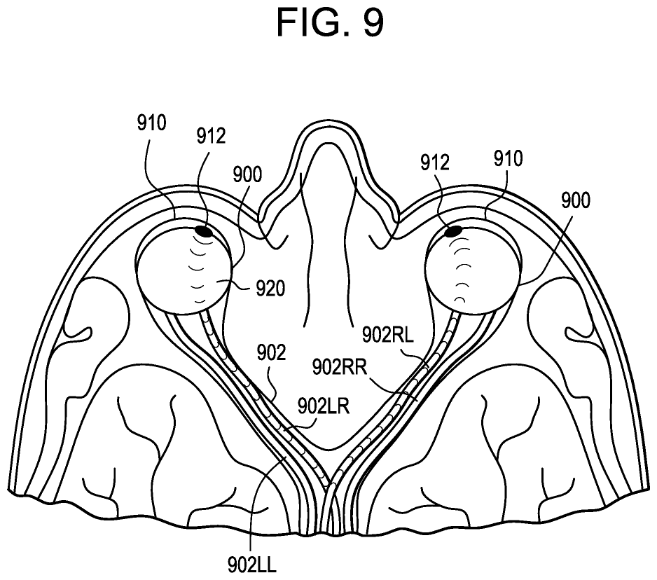

FIGS. 1-8 illustrate different embodiments according to the invention that include a system controller 130 that may be connected to a timing circuit 140 and an ultrasound module (collectively referred to as 110) that are on a contact lens 100. The ultrasound module 110 may take a variety of forms including distinct transmit and receive transducers or a shared transmit/receive transducer. The transducer(s) may face toward the optic nerve on the eye upon which the contact lens is worn. FIG. 9 illustrates a contact lens 910 with a transducer 912 placed on each eye 900, 900 where the transducers 912 are facing the optic nerve 902 (i.e., optic-nerve-facing), which is further divided into four nerves 902LL, 902LR, 902RL, and 902RR. For purposes of this disclosure and depending on the type of ophthalmic lens into which the device incorporated, the transducer(s) may be facing toward the optic nerve and toward the retina, the iris, and the ciliary muscle. In an intraocular lens embodiment, the transducer may be optic nerve facing. The sound pressure wave 920 produced by the ultrasound module will travel into the eye before propagating into the optic nerve 902LR to the optical Chiasm (the X-shaped structure formed at the point below the brain where the two optic nerves 902LR and 904RL cross over each other) where it will couple to the optic nerve 902RL of the other eye. Sound pressure wave 920 then continues travelling upon 902RL to the other eye then vibrating an iris-facing transducer (not illustrated) configured to receive the sound pressure wave on the other ophthalmic lens 910. This advantageously reduces the possibility of interference from possible environmental ultrasound waves and reflection of communication sound pressure waves that may be present. Experimental data demonstrate the viability of effectively propagating a sound pressure wave via the above-described pathway through the optical Chiasm. In other embodiments, there may be multiple ultrasound modules 110 present on the ophthalmic lens or, additionally or alternatively, multiple transducers connected to one or more ultrasound modules. Many of figures herein include an actuator 150 as part of the system with the actuator 150 being representative of, for example, lens accommodation components, data collection components, data monitoring components, and/or functional components such as an alarm.

An optic-nerve-facing transducer in combination with the path between the eyes through the optic nerve may provide a lower attenuation path, for example, than through use of scattering through the nose. Observed attenuation is an order of magnitude lower than through the use of air as the primary medium as would be the case were the transducers configured to face away from the eye. Lower attenuation lowers the voltage required to power the transducer to facilitate the communication through the eye. The use the optic nerve provides a unique pathway that decreases interference from other ultrasound sources.

System controller 130 in at least one embodiment uses at least one predetermined threshold for interpreting the output of the ultrasound module 110. In another embodiment, the system controller 130 makes use of at least one template (or pattern) to which a series of outputs of the ultrasound module 110 may be compared to determine whether the template has been satisfied. In an alternative embodiment, both thresholds and patterns are used by the system controller 130 to interpret a sound pressure waves. In at least one embodiment, as illustrated in FIG. 1, the system controller 130 is in electrical communication with a data storage device 132 that stores the threshold(s) and/or template(s). In at least one embodiment, a plurality of templates includes any combination of patterns and thresholds. Examples of data storage device 132 include memory such as persistent or non-volatile memory, volatile memory, buffer memory, a register(s), a cache(s), programmable read-only memory (PROM), programmable erasable memory, magneto resistive random-access memory (RAM), ferro-electric RAM, flash memory, and polymer thin film ferroelectric memory. In an alternative embodiment, the output(s) of the ultrasound module 110 to the system controller 130 is converted by the system controller 130 into data for control of the actuator 150. In an alternative embodiment, the system controller 130 interprets the output of the ultrasound module 110 using a predefined protocol.

FIG. 1 illustrates a system on a contact lens 100 having an electro-active region 102 with an ultrasound module 110, a system controller 130, an actuator 150, and a power source 180. In at least one further embodiment, the electro-active region 102 may circumscribe an optical zone of the lens. In other words, the electro-active region 102 may reside in a non-optic zone of the contact lens 100. Ultrasound module 110 in at least one embodiment may have two-way communication with system controller 130. Actuator 150 receives an output from system controller 130. In at least one alternative embodiment, actuator 150 is omitted from one or more of the illustrated embodiments in this disclosure.

Actuator 150 may include any suitable device for implementing an enabling a functionality upon receipt of a command signal from system controller 130. For example, system controller 130 may enable actuator 150 to change focus of a variable optic component, provide an alert to the wearer such as a light (or light array) to pulse a light into the wearer's retina (or alternatively across the lens), or to log data regarding the state of the wearer. In an alternative embodiment, actuator 150 sends an alert to an external device using, for example a forward-facing ultrasound module 110. Actuator 150 may receive a signal from system controller 130 in addition to power from the power source 180 and produce some action based on the signal from the system controller 130. For example, if the output signal from system controller 130 occurs during one operation state, then actuator 150 may alert the wearer that a medical condition has arisen, that lens is approaching the end of its useful life, or of another event that may be communicated from an external device, such as a smart device. In an alternative embodiment, actuator 150 delivers a pharmaceutical product to the wearer in response to an instruction from the system controller 130. In an alternative embodiment, the signal output by system controller 130 during another operation state may cause actuator 150 to record information in memory for later retrieval. In a still further alternative embodiment, a signal may cause the actuator to trigger an alarm and store information.

FIG. 1 illustrates a power source 180, which supplies power for numerous components in the system. The power may be supplied from a battery (a primary cell or rechargeable device), an energy harvester, solar cell, capacitor, or other suitable means as will be appreciated by one of ordinary skill in the art. Essentially, any type of power source 180 capable of providing reliable power for the components of the system may be utilized. In an alternative embodiment, communication functionality is provided by an energy harvester that acts as the receiver for a time signal, for example in an alternative embodiment, the energy harvester may be a photovoltaic cell, a photodiode, or a radio frequency (RF) receiver, which may receive both power and a time-based signal. In a further alternative embodiment, the energy harvester may be an inductive charger in which power is transferred in addition to data such as RFID. In one or more of these alternative embodiments, a time signal could be inherent in the harvested energy, for example N*60 Hz in inductive charging or lighting.

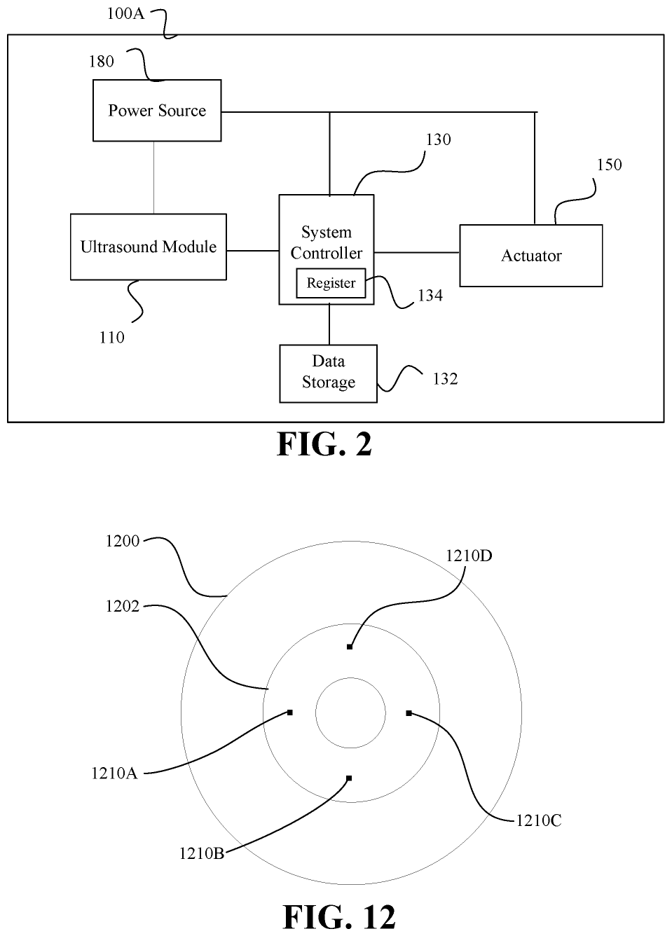

In at least one embodiment as illustrated in FIG. 2, contact lens 100A includes the system controller 130 having a register 134 for storing data samples from ultrasound module 110. In a further embodiment, an individual register may be used for each ultrasound module 110 and/or a receiving transducer present on contact lens 100A. The use of a register 134 in at least one embodiment allows for the comparison of data with prior data, a threshold, a preset value, a calibrated value, a target processing value, or a template with or without a mask. In an alternative embodiment, other data storage is used instead of a register(s). In an alternative embodiment, the register 134 is part of the data storage 132.

Based on this disclosure, it should be appreciated that in addition to the presence of the ultrasound module 110 on the contact lens 100 that additional sensors may be included as part of the contact lens to monitor characteristics of the eye and/or the lens. In at least one embodiment, at least a portion of the actuator 150 is consolidated with the system controller 130.

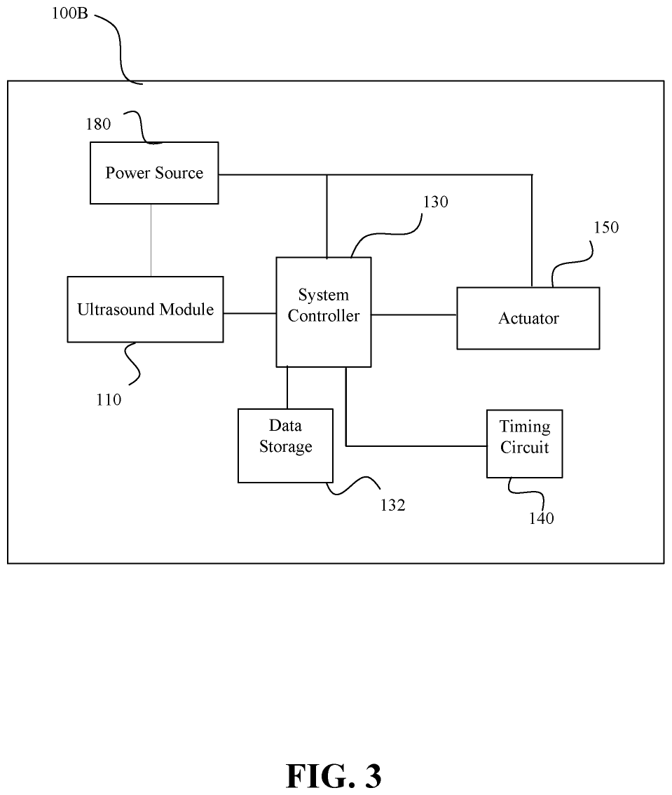

FIG. 3 illustrates another contact lens 100B that adds a timing circuit 140 to the system illustrated in FIG. 1. In an alternative embodiment, timing circuit 140 may also be added to the embodiment illustrated in FIG. 2. Timing circuit 140 provides a clock function for operation of the contact lens. As illustrated timing circuit 140 is connected to the system controller 130. In at least one embodiment, timing circuit 140 drives system controller 130 to send a signal to the ultrasound module 110 to perform a function based on a sampling time interval, which in at least one embodiment is variable based on the output from the ultrasound module 110 to system controller 130. In an alternative embodiment, timing circuit 140 may be part of the system controller 130.

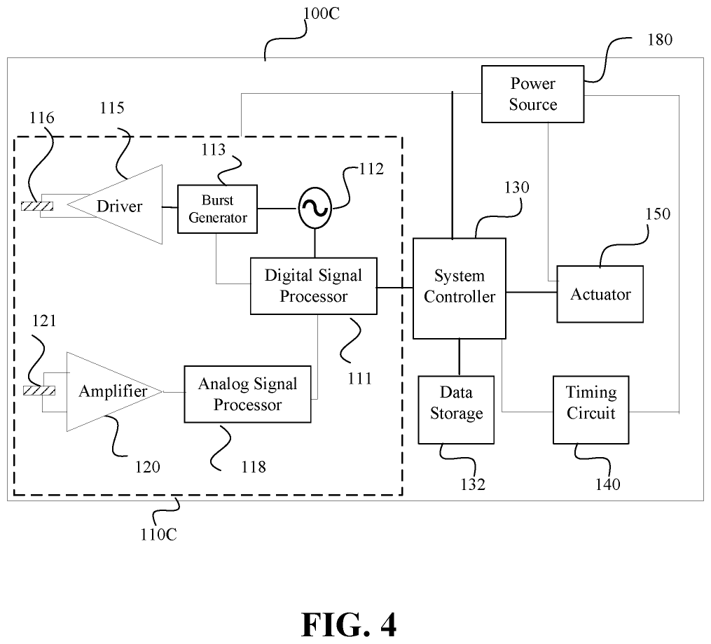

FIGS. 4-8 and 13 illustrate various ultrasound module embodiments that illustrate some exemplary transmit and receive paths that facilitate transmitting and receiving sound pressure waves from one or more transducers 116, 121 that start or end with a processor 111 and/or the system controller 130 depending on the example embodiment.

FIG. 4 illustrates a contact lens 100C that includes an ultrasound module 110C having distinct transmit and receive sides. The illustrated ultrasound module 110C includes a digital signal processor 111, an oscillator 112, a burst generator 113, a transmit driver 115, a transmit ultrasound transducer 116, an analog signal processor 118, a receive amplifier 120, and a receive ultrasound transducer 121. In at least one embodiment, the burst generator produces a series of 1's and 0's to facilitate communication with another lens. In at least one embodiment, burst generator 113 incorporates a unique identifier for the contact lens based on the amplitude, the frequency, the length, and/or the code modulation of the signal. In a further embodiment, the unique identifier is provided by the system controller 130, a digital signal processor 111, an oscillator 112, and/or a burst generator 113. In at least one alternative embodiment of ultrasound module 110C, digital signal processor 111 is combined with system controller 130. In another alternative embodiment, analog signal processor 118 may be combined with digital signal processor 111 and/or replaced with an analog-to-digital convertor as illustrated in a later figure.

Digital signal processor 111 may receive a control signal from system controller 130. In at least one embodiment, digital signal processor 111 includes a resettable counter and a time-to-digital convertor and transmit/receive sequencing controls. Oscillator 112 in at least one embodiment may take the form of a switched oscillator. In at least one embodiment, the frequency of oscillator 112 may be programmable via a preset oscillator value, the system controller 130 or external interface (e.g., an interface with an external device). The frequency can be tuned using a reference oscillator and an external interface. In at least one further embodiment, the frequency is set or tuned to a value that minimizes transmit and receive electrical power and allows the transmit ultrasound transducer 116 to produce a pressure sound wave that will have maximum amplitude at the receiver input. In a more particular embodiment, oscillator 112 may be a programmable frequency oscillator such as a current starved ring oscillator where current and capacitance control the oscillation frequency, which may be altered by changing the current supplied to the oscillator. In at least one embodiment, the wavelength of the sound pressure wave is tuned based on the dimensions of the transducer used. In a further embodiment, the oscillator 112 varies over time for optimal transmission characteristics. In a still further embodiment, the frequency is calibrated using a reference frequency provided through an external interface and an automatic frequency control (AFC) circuit. The frequency may be preset with the AFC tuning it. The frequency may be directly set through the serial interface, which may be accessed through an external communications link.

An output voltage of burst generator 113 may be level shifted to the appropriate value for transmit driver 115 and transmit ultrasound transducer 116. An example of the transmit ultrasound transducer 116 is a piezoelectric device that converts an applied burst voltage to a sound pressure burst. In a further embodiment, transmit ultrasound transducer 116 may be composed of any piezoelectric material compatible with the power source and the physical properties of the contact lens. Some non-limiting examples of possible piezoelectric materials that may be utilized include PZT: Lead zirconate titanate (types 100, 200, 300, 500, 600) PT: Lead titanate (type 700) PMN: Lead metaniobate (type 800) PVDF: Polymer P(VDF-TrFE): Copolymer BT: Bismuth titanate PSC: Piezoelectric single crystals 1-3 composites: PZT and polymer and similar materials and composites thereof. A sound pressure wave produced by transmit ultrasound transducer 116 propagates from the contact lens 100 into the eye.

Receive amplifier 120 and analog signal processor 118 in at least one embodiment are turned on by oscillator 112 or turned on after a predetermined delay after oscillator 112 is started. When there is a predetermined delay, power for the contact lens operation may be reduced during the period of delay. In an embodiment where receive amplifier 120 and analog signal processor 118 are started by oscillator 112, receive amplifier 120 may receive an output from receive ultrasound transducer 121 contemporaneously with the sound pressure wave being output by transmit ultrasound transducer 116. This output from receive ultrasound transducer 121 may be used to reset a counter in digital signal processor 111. In a further embodiment, detecting a transmit sound pressure wave may serve as an indicator that a true transmit signal has been generated.

A sound pressure wave received by receive ultrasound transducer 121 may produce a voltage signal having frequency and burst length properties related to the transmitted sound pressure wave. The voltage signal may be amplified by receive amplifier 120 before being sent to analog signal processor 118. Analog signal processor 118 may include, but is not limited to, frequency-selective filtering, envelope detection, integration, level comparison and/or analog-to-digital conversion. Based on this disclosure, it should be appreciated that these functions may be separated into individual blocks with some examples being illustrated in later figures. Analog signal processor 118 produces a received signal that represents the received sound pressure wave at the receive ultrasound transducer 121, which in implementation will have a slight delay. The received signal is passed from analog signal processor 118 to digital signal processor 111. The resulting output from digital signal processor 111 may be provided to the system controller 130.

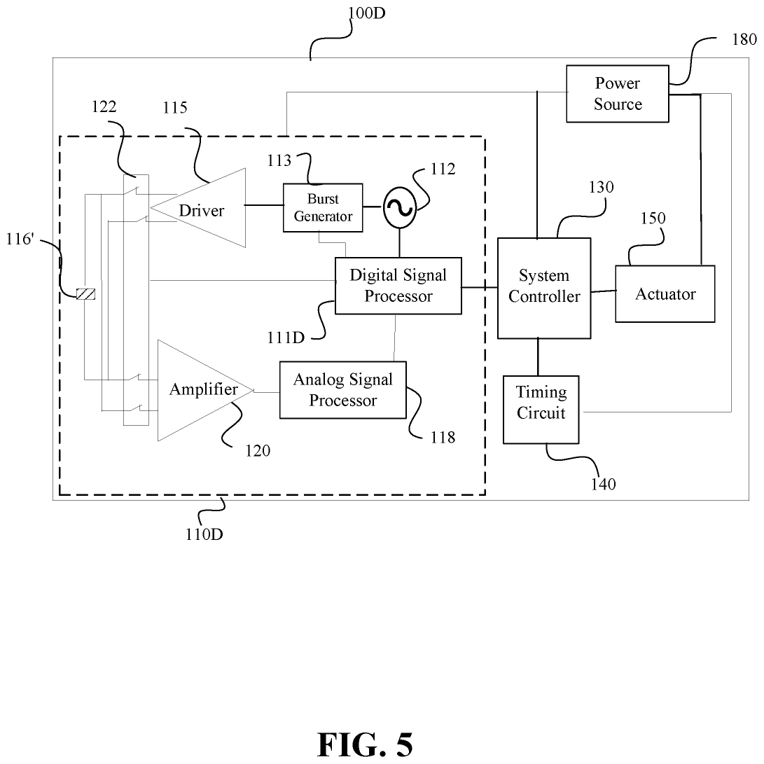

FIG. 5 illustrates a contact lens 100D including an ultrasound module 110D. Ultrasound module 110D includes ultrasound transducer 116′, which may be shared by transmit and receive sides (or paths). Single ultrasound transducer 116′ may be multiplexed between transmit and receive operations through use of a switch 122. Digital signal processor 111D may use the output of burst generator 113 to switch transducer 116′ to a transmit mode by connecting transmit driver 115 to transducer 116′. When a burst is completed, then the digital signal processor 111D may toggle switch 122 to receive mode by connecting receive amplifier 120 to transducer 116′. One advantage to this configuration is that the transducer area is reduced from two transducers to one. As with the previous embodiment, a delay may be (although not necessary in all embodiment) imposed after transmission before the receive amplifier 120 is powered. The remaining components of the illustrated embodiment remain the same from the prior embodiment.

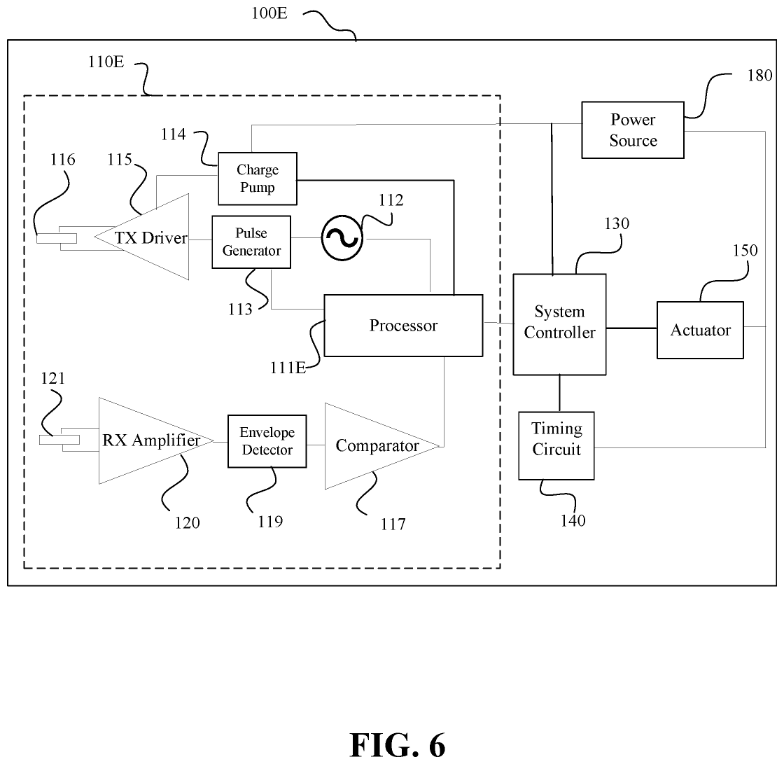

FIG. 6 illustrates a contact lens 100E with an ultrasound module 110E. The illustrated ultrasound module 110E includes a processor 111E, oscillator 112, pulse generator 113, charge pump 114, transmit driver 115, transmit ultrasound transducer 116, comparator 117, envelope detector 119, receive amplifier 120, and receive ultrasound transducer 121. The charge pump 114 is electrically connected to power source 180 and to transmit driver 115, which provides a voltage to transmit ultrasound transducer 116 to create a sound pressure wave to be emitted by the transducer 116. In at least one embodiment, transmit driver 115 includes an inverter or an H-bridge, and in further embodiments includes an output driver circuit. In at least one embodiment, charge pump 114 increases the voltage through the relationship between charge and capacitance with voltage by increasing the charge on a capacitance component(s) (e.g., a capacitor). The voltage output from the charge pump 114, in at least one embodiment, is used as the supply voltage to the transmit driver 115. Transmit driver 115 switches between the output of charge pump 114 and ground in an alternating fashion in response to the input from pulse generator 113 to produce an alternating voltage. The alternating voltage is applied by the driver 115 to polarize the material of the transducer 116 in one direction and then the other direction to create a mechanical stress causing the material to be displaced in a specific direction (i.e. the direction the transducer is facing). The displacement of the transducer material coupled with the shape and the size of the transducer produce the sound pressure wave. Thus, the larger the applied voltage is to the transducer, the larger the stress and thus the larger the displacement and associated sound pressure wave.

Charge pump 114 is also electrically connected to processor 111E, which controls operation of charge pump 114 in at least one embodiment to minimize power consumption by the system by, for example turning off oscillator 112, pulse generator 113, and/or charge pump 114 at times when the ultrasound module 110E does not need to propagate a sound pressure wave. An envelope detector 119 turns the high-frequency output of receive ultrasound transducer 121 into a new signal that provides an envelope signal representative of the original output signal to be provided to comparator 117. This illustrated embodiment has the advantage of simplifying the analysis of the output of receive ultrasound transducer 121 to determine if a particular threshold has been met for the contact lens 100E to perform a function. Comparator 117 provides an output to the processor 111E, which is in electrical communication with the system controller 130.

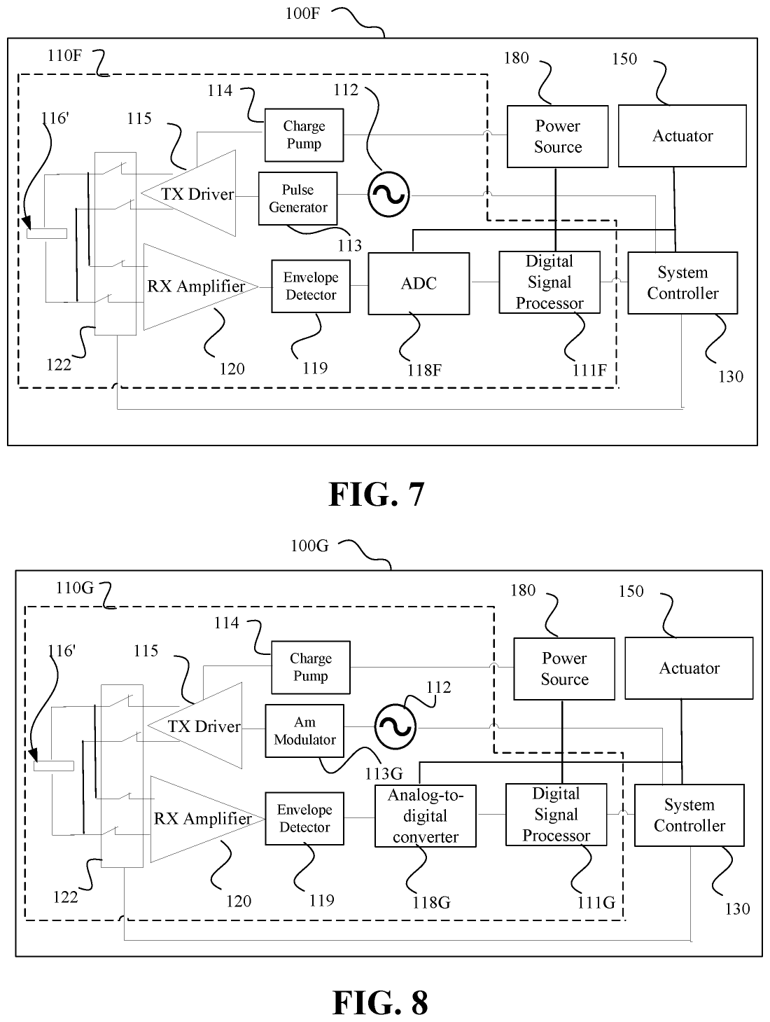

FIG. 7 illustrates a contact lens 100F with an ultrasound module 110F. The illustrated ultrasound module 110F includes a digital signal processor 111F, an oscillator 112, a pulse generator 113, a charge pump 114, a transmit driver 115, a transmit/receive ultrasound transducer 116′, an analog-to-digital converter (ADC) 118F, an envelope detector 119, a receive amplifier 120, and a switch 122. The ADC 118F converts an output from envelope detector 119 into a digital signal for digital signal processor 111F.

FIG. 8 illustrates a contact lens 100G with an ultrasound module 110G. The illustrated ultrasound module 110G includes a digital signal processor 111G, the oscillator 112, an amplitude modulation (AM) modulator 113G, a charge pump 114, a transmit driver 115 such as a transmit amplifier, a transmit/receive ultrasound transducer 116′, an analog-to-digital converter (ADC) 118G, an envelope detector 119, a receive ultrasound transducer 121, and a switch 122. In the illustrated embodiment, charge pump 114, AM modulator 113G and transmit driver 115 act as a level shifter and burst generator. AM modulator 113G in this embodiment is controlled by digital signal processor 111G. The circuit may operate to cause an oscillator signal to be provided to AM modulator 113G, which in at least one embodiment may be an AND gate, and digital signal processor 111G provides a second clock at a frequency that may be lower, and in some cases substantially lower, than the oscillator frequency. The output of the circuit may then take the form of a sequence of pulses that occur during the positive cycle of the lower frequency. The transmit driver 115 may be configured with an appropriate gain to output the modulated signal at the charge pump voltage thus providing level shifting.

Based on the disclosure connected to FIGS. 6-8, one of ordinary skill in the art should appreciate that the different ultrasound module configurations and transducer/switch configurations may be interchanged and mixed together in different combinations.

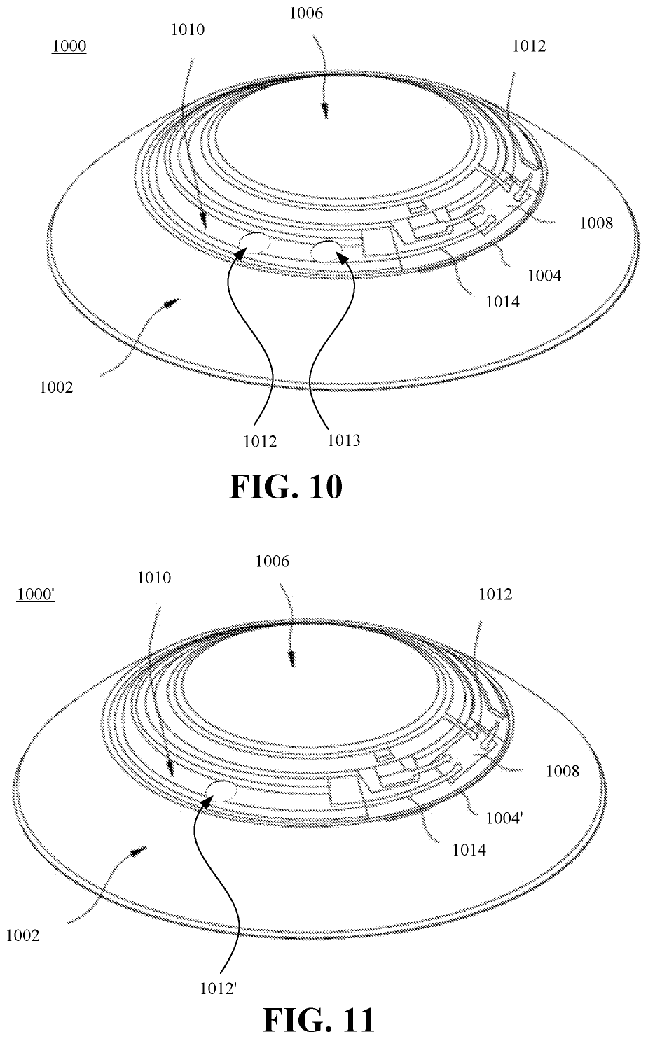

FIG. 10 illustrates a contact lens 1000 with an electronic insert 1004 having an ultrasound module. Contact lens 1000 may include a soft plastic portion 1002 that houses an electronic insert 1004, which in at least one embodiment may include an electronics ring around lens 1006. This electronic insert 1004 may include a variable lens 1006 which is activated by the electronics, for example focusing near or far depending on activation (or accommodation level). Integrated circuit 1008 mounts onto the electric insert 1004 and connects to batteries (or other power source) 1010, variable lens 1006, and other components as desired for the system.

In at least one embodiment, a transmit ultrasound transducer 1012 and a receive ultrasound transducer 1013 are present in the ultrasound module. In at least one embodiment, the integrated circuit 1008 includes a transmit ultrasound transducer 1012 and a receive ultrasound transducer 1013 with the associated signal path circuits. Transducers 1012, 1013 may face inwards towards the optic nerve of the wearer (i.e. optic nerve facing) through the lens insert and towards the eye, and thus may be able to send and receive sound pressure waves into the eye for propagation into the optic nerve and the other eye for delivery to the receive transducer of the other contact lens. In at least one embodiment, transducers 1012, 1013 are fabricated separately from the other circuit components in the electronic insert 1004 including the integrated circuit 1008. In this embodiment, transducers 1012, 1013 may also be implemented as separate devices mounted on the insert 1004 and connected with wiring traces 1014. Alternatively, transducers 1012, 1013 may be implemented as part of the integrated circuit 1008 (not shown). Based on this disclosure one of ordinary skill in the art should appreciate that transducers 1012, 1013 may be augmented by the other sensors.

FIG. 11 illustrates another contact lens 1000′ with an electronic insert 1004′ having an ultrasound module. The contact lens 1000′ may include a soft plastic portion 1002 that houses the electronic insert 1004′. This electronic insert 1004′ may include a variable lens 1006 which is activated by the electronics, for example focusing near or far depending on activation (or accommodation level). Integrated circuit 1008 may mount onto electronic insert 1004′ and may connects to batteries (or other power source) 1010, lens 1006, and other components as desired for the system. The ultrasound module includes a transmit/receive ultrasound transducer 1012′ with the associated signal path circuits. The transducer 1012′ may inward through the lens insert and towards the optic nerve of the eye, and thus may be able to send and receive sound pressure waves. As discussed above, transducer 1012′ may be fabricated separately from the other electronic components prior to mounting on electronic insert 1004 or alternatively implemented on integrated circuit 1008 (not shown). Transducer 1012′ may also be implemented as a separate device mounted on the electronic insert 1004′ and connected with wiring traces 1014. Based on this disclosure one of ordinary skill in the art should appreciate that transducer 1012′ may be augmented by the other sensors.

In a further embodiment to the embodiments illustrated in FIGS. 10 and 11, integrated circuit 1008, power source 1010 and transducers 1012, 1012′, 1013 may be disposed within an area of the contact lens contained in an overmold, which is a material (such as plastic or other protective material) that encapsulates electronic insert 1004. In at least one further embodiment, the overmold encapsulates the ultrasound module(s).

In at least one embodiment as illustrated in FIG. 12 (omits several components to facilitate presentation clarity), there may be a plurality of ultrasound modules 1210A-1210D spaced around the contact lens 1202 on eye 1200 to increase the fidelity of the communication link between the contact lenses through the nose. Although four ultrasound modules 1210A-1210D are illustrated, it should be appreciated based on this disclosure that a variety of numbers of ultrasound modules may be used with example numbers of ultrasound modules being any number between 2-8, a plurality of ultrasound modules, and at least one ultrasound module. The illustrated ultrasound modules 1201A-1210D may be evenly spaced around the periphery of the contact lens 1202 where evenly spaced includes equal distance between the ultrasound modules (i.e., the same distance between neighboring ultrasound modules) and/or balanced about a diameter drawn through the contact lens 1202.

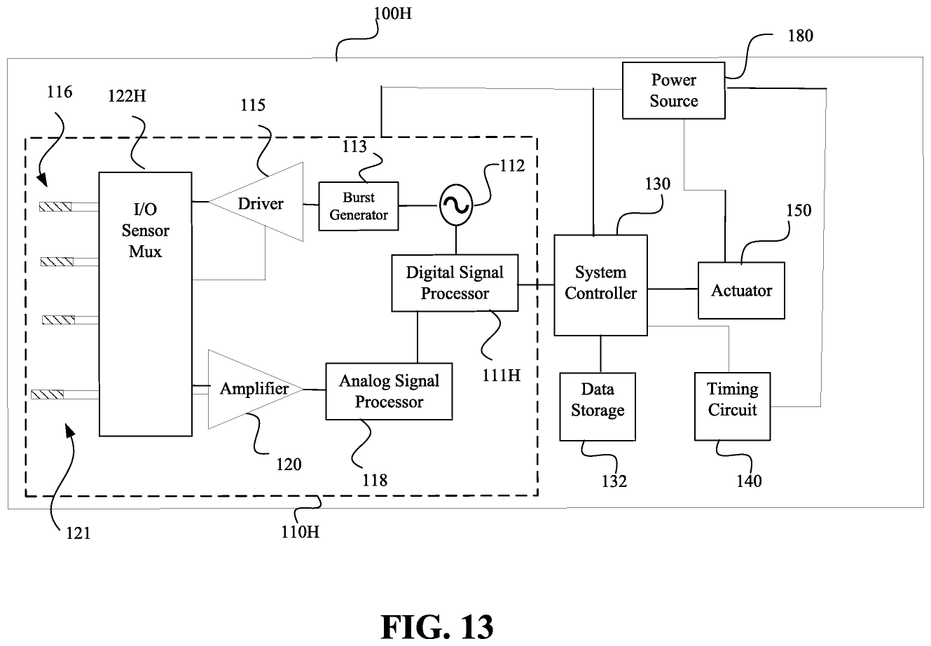

In at least one embodiment, the system controller deactivates the transmission components of the ultrasound module when the respective contact lens is not transmitting. In a further embodiment, the illustrated ultrasound modules may be replaced by transducers that are multiplexed together as illustrated in FIG. 13. In a further embodiment for contact lenses that have a plurality of ultrasound modules or at least a plurality of transmit/receive/transceiver transducers, the method includes having the system controller determine which ultrasound module/transducer provides the best response. The system controller selects the ultrasound module/transducer that produces a highest output response to received sound pressure waves. The system controller will deactivate the ultrasound module(s)/transducer(s) that were not selected (i.e., provided a lower signal strength).

In an alternative embodiment illustrated in FIG. 13, contact lens 100H has one ultrasound module 110H having a plurality of transducers 116, 121 and an I/O multiplexer (mux) 122H attaching the transducers 116, 121 to the ultrasound module components discussed in the above embodiments. FIG. 13 illustrates the inclusion of digital signal processor 111H, the oscillator 112, burst generator 113, driver 115, amplifier 120, the analog signal processor 118. In an alternative embodiment, these ultrasound module components may be replaced by components from the other described ultrasound module embodiments including using just the transmit or receive paths of those embodiments. An advantage of this configuration is that it may reduce the power requirements and weight considerations by eliminating duplicative components and allowing the ultrasound module to drive multiple transmit transducers and to receive analog signals from multiple receive transducers. In at least one embodiment, the transmit transducers and the receive transducers are distributed about the contact lens as discussed above in connection with FIG. 12. In a further embodiment, the transmit transducers and the receive transducers may be grouped together in one area of the contact lens.

In at least one embodiment where the contact lens includes rotational stability features, then the number of ultrasound modules is one.

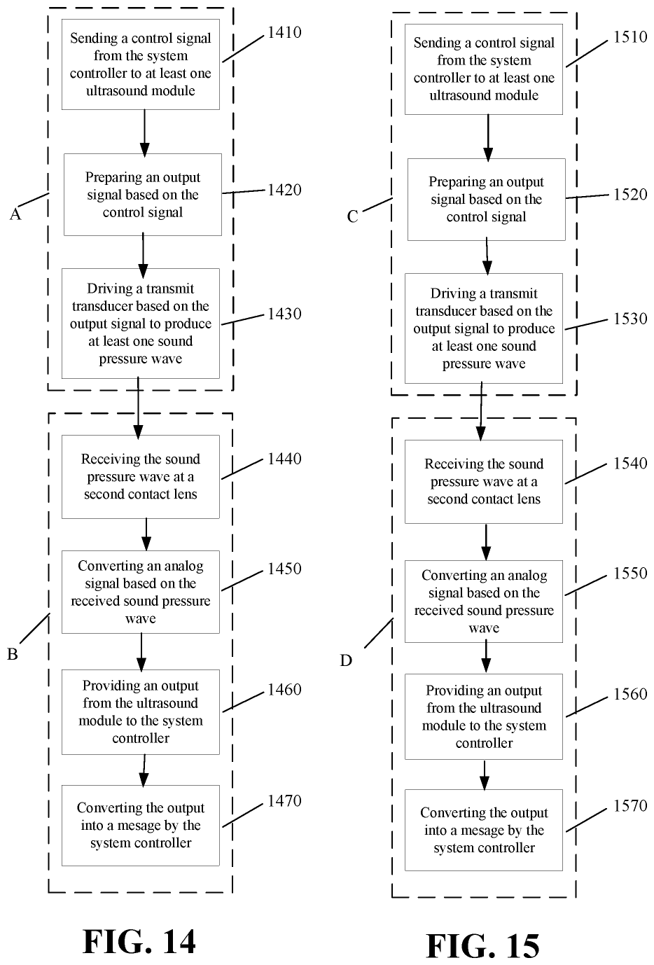

FIG. 14 illustrates a method that may be used with more than one of the above-described system embodiments. The illustrated method provides an example of how communication may be facilitated between two contact lenses, a first contact lens and a second contact lens, through the eyes and an optic nerve of the person wearing (or using) the contact lenses. The eyes and the optic nerve provides a medium in which the sound pressure waves produced by at least one transducer on one contact lens travels towards the other contact lens. FIG. 14 is divided into two groups of steps A and B to indicate which lens performs the respective steps (i.e., the first contact lens A and the second contact lens B). In at least one embodiment, similar methods can be used for implanted intraocular lenses during use.

The system controller on the first contact lens sends a control signal that embodies a message intended for the second contact lens to the ultrasound module(s), 1410. The message may include sensor data, a request for sensor data, a request for confirmation of data interpretation (e.g., direction of focus and/or contact lens orientation), data interpretation, an instruction to perform a function such as with the actuator and/or a predefined function, etc. In at least one embodiment, the message is created by the system controller using a predetermined protocol for communication between the contact lenses. The ultrasound module prepares an output signal based on control signal 1420. In an alternative embodiment, the output signal preparation is omitted if the control signal is sufficient for driving the transducer, which may be a dedicated transmit transducer. The ultrasound module drives the transducer to produce at least one sound pressure wave based on output signal 1430.

The second contact lens receives the at least one sound pressure wave from the first contact lens propagated through the eyes and the optic nerve, 1440. The second contact lens uses its transducer, which in at least one embodiment is a dedicated receive transducer. When the contact lens(es) has a common transceiver transducer to transmit and receive, then in at least one embodiment the transceiver transducer is in a default position of receive mode. The ultrasound module converts an analog signal representing received sound pressure wave received by the transducer, 1450. The resulting output is provided by the ultrasound module to a system controller, 1460. The system controller converts the output into the message from the system controller on the first contact lens, 1470.

FIG. 15 illustrates a method for the second contact lens to respond to the first contact lens. FIG. 15 is divided into two groups of steps C and D to indicate which lens performs the respective steps (i.e., the second contact lens C and the first contact lens D).

The system controller on the second contact lens sends a control signal that embodies a message intended for the first contact lens to the ultrasound module(s), 1510. The message may include sensor data, a request for sensor data, a request for confirmation of data interpretation (e.g., direction of focus and/or contact lens orientation), data interpretation, an instruction to perform a function such as with the actuator, etc. The ultrasound module prepares an output signal based on the control signal, 1520. In an alternative embodiment, the output signal preparation is omitted if the control signal is sufficient for driving the transducer, which may be a dedicated transmit transducer. The ultrasound module drives the transducer to produce at least one sound pressure wave based on the output signal, 1530.

The first contact lens receives the at least one sound pressure wave from the second contact lens through the eyes and the optic nerve, 1540. The first contact lens uses its transducer, which in at least one embodiment is a dedicated receive transducer. When the contact lens have a common transceiver transducer to transmit and receive, then in at least one embodiment the transceiver transducer is in a default position of receive mode. The ultrasound module converts an analog signal representing received sound pressure wave received by the transducer, 1550. The resulting output is provided by the ultrasound module to a system controller, 1560. The system controller converts the output into the message from the system controller on the second contact lens, 1570. In a further embodiment, the first contact lens performs a function based on the received message such as change the activation level of the lens.

In an alternative embodiment to the methods illustrated in FIGS. 14 and 15, the system controller deactivates the transmission components of the ultrasound module when the respective contact lens is not transmitting.

In a further embodiment, the sound pressure waves produced by the first and second contact lenses are at different frequencies such as the first contact lens using a first frequency and the second contact lens using a second frequency. The ultrasound module in at least one embodiment then is tuned for the frequency of the output sound pressure wave produced by the other contact lens. An advantage of this is that it improves each receiver's capability of correctly detecting the desired signal. By using separate frequencies, frequency selective techniques (such as mixing and envelope detection) can reject noise or scattered undesired transmit signals that could be produced by the physical geometry and properties of the communication channel through the contact lens, eye and optic nerve.

In a further embodiment, the message sent is a wake-up message to activate the ultrasound module(s) on the second contact lens. In at least one implementation, the second contact lens will activate for short periods of time at a predetermined sampling rate to detect the wake-up message being broadcasted by the first contact lens at a predetermined broadcast rate. In at least one embodiment the predetermined sampling rate and the predetermined broadcast rate are at different frequencies where one rate is faster than the other to allow for the sampling and the broadcasting to intersect eventually. Alternatively, the short period of time is of sufficient length to cover the frequency period for the predetermined broadcast rate or slightly longer to address a situation where the clock frequencies of the two contact lenses may be different. A wake-up message can be used for initial activation of the second contact lens along with reactivation of the second contact lens, for example when the contact lenses are in a slower operational or sleep state when the wearer is asleep or resting or alternatively has set the operational mode to a state in which communication between the contact lenses is not necessary. In a further embodiment, the wake-up message is sufficient strength and length to facilitate the second contact lens generating sufficient power to activate in response to the wake-up message such as the energy harvester being activated by the current generated by the receive transducer.

In a further embodiment for contact lenses that have a plurality of ultrasound modules or at least transmit/receive/transceiver transducers, the method includes having the system controller determine which ultrasound module/transducer provides the best communication path. The system controller selects the ultrasound module/transducer that produces a highest output response to the sound pressure wave produced by the other contact lens. This measurement may be made during performance of the above-described communication methods or a communication consisting of pinging back and forth between the contact lens. The pinging communication may occur on a predetermined schedule or at predetermined intervals possibly even as part of a clock synchronization between the contact lenses. The system controller will deactivate the ultrasound module(s)/transducer(s) that were not selected (i.e., provided a lower signal strength). One benefit to this method is that as the contact lens rotates on the eye, the system controller can change the used ultrasound module/transducer for intra-contact communication.

One approach to facilitate the communication between the contact lenses is to implement automatic frequency control for the communication channel. In at least one embodiment, the timing circuit on one contact lens would be the master. The clock synchronization in at least one embodiment will lead the electronics to be biased towards a lens pair to have one be a master. In a further embodiment, the selection of the master contact lens is made post-manufacturing via a software download to the lenses and/or settings change. This approach also could be used to facilitate the dual frequency approach discussed in this disclosure.

Although shown and described in what is believed to be the most practical embodiments, it is apparent that departures from specific designs and methods described and shown will suggest themselves to those skilled in the art and may be used without departing from the spirit and scope of the invention. The present invention is not restricted to the particular constructions described and illustrated but should be constructed to cohere with all modifications that may fall within the scope of the appended claims.

What is claimed is:

1. An ophthalmic lens system comprising:
a first ophthalmic lens;
a second ophthalmic lens; and
wherein each of the first and second ophthalmic lenses comprises:
an ultrasound module including at least one ultrasound transducer disposed within the ophthalmic such that such that a sound pressure wave produced by the at least one transducer travels through an eye upon which the ophthalmic lens worn to an optical nerve connected to the eye; and
a system controller in electrical communication with the ultrasound module, wherein the system controller is configured to:
provide a control signal comprising a message to the ultrasound module, wherein the control signal includes a message to be transmitted by the ultrasound module; and
receive an output from the ultrasound module and to perform a function in response to a received message embodied in the output.

2. The ophthalmic lens systems according to claim 1, wherein the first ophthalmic lens is a first contact lens and the second ophthalmic lens is a second contact lens.

3. The ophthalmic lens systems according to claim 1, wherein the first ophthalmic lens is a first intraocular lens and the second ophthalmic lens is a second intraocular lens.

4. The ophthalmic lens system according to claim 1, wherein:
the ultrasound module on the first ophthalmic lens is configured to produce the sound pressure wave at a first frequency,
the ultrasound module on the second ophthalmic lens configured to produce the sound pressure wave at a second frequency,
the ultrasound module on the second ophthalmic lens has a receive transducer tuned to sense the sound pressure wave at the first frequency, wherein the receive transducer is optic-nerve facing, and
the ultrasound module on the first ophthalmic lens has a receive transducer tuned to sense the sound pressure wave at the second frequency, wherein the receive transducer is optic-nerve facing.

5. The ophthalmic lens system according to claim 4, wherein
the ultrasound module on the first ophthalmic lens has a second receive transducer tuned to sense the sound pressure wave at the first frequency, and
the ultrasound module on the second ophthalmic lens has a second receive transducer tuned to sense the sound pressure wave at the second frequency, and
the second receive transducers are optic nerve facing.

6. The ophthalmic lens system according to claim 1, wherein at least one of the ultrasound modules are disposed near a perimeter of the ophthalmic lens in which the ultrasound modules are disposed.

7. The ophthalmic lens system according to claim 1, wherein
the at least one ultrasound transducer includes a transmit transducer and a receive transducer, and
each ultrasound module includes
a processor in electrical communication with the system controller;
a transmit path having:
an oscillator in electrical communication with the processor,
a burst generator in electrical communication with the oscillator and the processor,
a transmit driver in electrical communication with the burst generator, wherein the transmit driver is configured to receive a burst signal from the burst generator,
the transmit transducer in electrical communication with the transmit driver; and
at least one receive path including:
the receive transducer,
a receive amplifier in electrical communication with the receive transducer and configured to amplify an output of the receive transducer, and
an analog signal processor in communication with the receive amplifier and the processor, and
wherein the processor is configured to control whether one or both of the transmit path and the at least one receive path are activated.

8. The ophthalmic lens system according to claim 7, wherein each ultrasound module includes two receive paths,
the two receive paths having the receive transducer tuned to different frequencies.

9. The ophthalmic lens system according to claim 1, wherein
the at least one transducer includes a plurality of transducers, and
the ultrasound module includes
a processor in electrical communication with the system controller;
a multiplexer in electrical communication with the plurality of transducers;
a transmit path having:
an oscillator in electrical communication with the processor,
a burst generator in electrical communication with the oscillator and the processor,
a transmit driver in electrical communication with the burst generator, wherein the transmit driver is configured to receive a burst signal from the burst generator and the multiplexer; and
at least one receive path having:
a receive amplifier in electrical communication with the multiplexer and configured to amplify an output of the receive transducer, and
an analog signal processor in communication with the receive amplifier and the processor, and
wherein the processor is configured to control whether one or both of the transmit path and the at least one receive path are activated, and
the multiplexer provides selective communication between at least one transducer with the transmit path or the at least one receive path.

10. The ophthalmic lens system according to claim 1, wherein
the at least one transducer includes one transducer, and
each ultrasound module includes:
a processor in electrical communication with the system controller;
the transducer;
a switch in electrical communication with the processor;
a transmit path having:
an oscillator in electrical communication with the processor,
a burst generator in electrical communication with the oscillator and the processor,
a transmit driver in electrical communication with the burst generator, wherein the transmit driver is configured to receive a burst signal from the burst generator, the transmit driver drives the transducer when connected through the switch; and
at least one receive path having:
a receive amplifier in electrical communication with the transducer through the switch and configured to amplify an output of the transducer, and
an analog signal processor in communication with the receive amplifier and the processor, and
wherein the processor is configured to control whether one or both of the transmit path and the at least one receive path are activated based on an operation mode of the ultrasound module between transmit and receive, and
the processor configured to control the switch and the operation mode.

11. The ophthalmic lens system according to claim 1, wherein each ophthalmic lens further includes a power source in electrical communication with the system controller and the ultrasound module;
wherein the at least one transmitter includes a transmit transducer and a receive transducer; and
each ultrasound module includes:
a processor in electrical communication with the system controller;
a transmit path having:
an oscillator in electrical communication with the processor,
a pulse generator in electrical communication with the oscillator and the processor,
a charge pump in electrical communication with the power source,
a transmit driver in electrical communication with the pulse generator and the charge pump, wherein the transmit driver is configured to receive a signal from the pulse generator,
the transmit transducer being in electrical communication with the transmit driver; and
at least one receive path having:
the receive transducer,
a receive amplifier in electrical communication with the receive transducer and configured to amplify an output of the receive transducer, and
an envelope detector in electrical communication with the receive amplifier,
an analog signal processor in communication with the envelope detector and the processor, and
wherein the processor is configured to control whether the transmit path and the at least one receive path are activated.

12. The ophthalmic lens system according to claim 1, wherein each ophthalmic lens further includes a power source in electrical communication with the system controller and the at least one ultrasound module;
the at least one transmitter includes a transmit transducer and a receive transducer; and
each ultrasound module includes
a processor in electrical communication with the system controller;
a transmit path having
an oscillator in electrical communication with the processor,
an amplitude modulation modulator in electrical communication with the oscillator and the processor,
a charge pump in electrical communication with the power source,
a transmit driver in electrical communication with the amplitude modulation modulator and the charge pump, wherein the transmit driver is configured to receive a signal from the amplitude modulation modulator,
wherein the transmit transducer is in electrical communication with the transmit driver; and
at least one receive path having:
the receive transducer,
a receive amplifier in electrical communication with the receive transducer and configured to amplify an output of the receive transducer, and
an envelope detector in electrical communication with the receive amplifier,
an analog signal processor in communication with the envelope detector and the processor, and
wherein the processor is configured to control whether the transmit path and the at least one receive path are activated.

13. A method for facilitating communication between a first ophthalmic lens and a second ophthalmic lens when being worn by a person where each ophthalmic lens includes at least one ultrasound module in electrical communication with a system controller, the ultrasound modules having an iris-facing transmit transducer, the method comprising:
sending a control signal from the system controller on the first ophthalmic lens to the ultrasound module on the first ophthalmic lens wherein the control signal embodies a message intended for the second ophthalmic lens;
preparing an output signal by the ultrasound module on the first ophthalmic lens based on the message;
driving the transmit transducer on the first ophthalmic lens based on the output signal to produce at least one sound pressure wave directed at the eye to travel through an optical nerve to the other eye;
receiving with a transducer on the second ophthalmic lens the sound pressure wave from the transducer on the first ophthalmic lens transmitted through the optical nerve;
converting with the ultrasound module on the second ophthalmic lens an analog signal produced by the transducer on the second ophthalmic lens in response to the received sound pressure wave;
providing an output to the system controller on the second ophthalmic lens from the ultrasound module on the second ophthalmic lens; and
converting with the system controller on the second ophthalmic lens the output into the message from the system controller on the first ophthalmic lens, and
wherein the optical nerve and the eyes of the person wearing the ophthalmic lenses provides a medium through which the sound pressure wave travels between the first ophthalmic lens and the second ophthalmic lens.

14. The method according to claim 13, further comprising:
sending a control signal from the system controller on the second ophthalmic lens to the ultrasound module on the second ophthalmic lens where the control signal embodies a message intended for the first ophthalmic lens;
preparing an output signal by the ultrasound module on the second ophthalmic lens, where the output signal embodies the message for the first ophthalmic lens based on the message intended for the first ophthalmic lens;
driving the transmit transducer on the second ophthalmic lens based on the output signal to produce at least one sound pressure wave directed at the eye to travel through an optical nerve to the other eye;
receiving with a receive transducer on the first ophthalmic lens sound pressure wave from the transducer on the second ophthalmic lens transmitted through the optical nerve;
converting with the ultrasound module on the first ophthalmic lens an analog signal produced by the receive transducer on the first ophthalmic lens;
providing an output to the system controller on the first ophthalmic lens from the ultrasound module on the first ophthalmic lens; and
converting with the system controller on the first ophthalmic lens the output into the message from the system controller on the first ophthalmic lens, and
wherein the optical nerve and the eyes of the person wearing the ophthalmic lenses provides the medium through which the sound pressure wave travels between the second ophthalmic lens and the first ophthalmic lens.

15. The method according to claim 14, wherein the sound pressure waves produced by the first and second ophthalmic lens are at different frequencies.

16. The method according to claim 15, wherein each ultrasound module includes the receive transducer tuned to the frequency of the transmit transducer of the other ophthalmic lens and a second receive transducer tuned to the frequency of the transmit transducer of its ophthalmic lens.

17. The method according to claim 13, wherein the method of operation of one ophthalmic lens further comprising:
sampling the receive transducer at a first sampling rate;
sampling the receive transducer at a second sampling rate when the sound pressure wave is detected by the system controller based on the output from the receive transducer;
determining with the system controller when the message being received is complete; and
sampling the receive transducer at the first sampling rate when the system controller determines completion of the message being sent.

18. The method according to claim 13, further comprising deactivating the transmission components of the ultrasound module when not transmitting.

19. The method according to claim 13, wherein the message sent by the system controller of the first ophthalmic lens uses a predefined protocol.

20. The method according to claim 13, wherein the message sent by the system controller of the first ophthalmic lens includes instructions for the second ophthalmic lens to perform a predefined function.

21. The method according to claim 20, wherein the predefined function includes providing a sensor reading where the sensor reading will be used by the system controller on the first ophthalmic lens to confirm interpretation of at least one sensor reading on the first ophthalmic lens; and
the method further comprising
the system controller on the second ophthalmic lens sending the sensor reading in a message provided to the respective ultrasound module, and
producing a sound pressure wave with the transmit transducer of the second ophthalmic lens applied to the eye on which the second ophthalmic lens is located where the sound pressure wave travels through the optical nerve to the first ophthalmic lens.

22. The method according to claim 13, wherein the message sent by the system controller of the first ophthalmic lens includes sensor readings from at least one sensor on the first ophthalmic lens.

23. The method according to claim 13, wherein the message is a wake-up message to activate the ultrasound module on the second ophthalmic lens.
